(19) United States
(12) Patent Application Publication
(10) Pub. No.: US 2023/0263933 A1
(43) Pub. Date: Aug. 24, 2023

(54) WOUND HYDROGEL FOR MANAGING ACUTE AND CHRONIC WOUNDS IN HUMAN AND ANIMALS

(71) Applicant: V3BIO SDN. BHD., Bandar Cassia (MY)

(72) Inventor: Kamaleswaran A/L RAMACHANDRAM, Bandar Cassia (MY)

(73) Assignee: V3BIO SDN. BHD., Bandar Cassia (MY)

(21) Appl. No.: 18/112,869

(22) Filed: Feb. 22, 2023

(30) Foreign Application Priority Data
Feb. 23, 2022 (MY) PI 2022001024

(51) Int. Cl. A61L 26/00

(52) U.S. Cl. CPC A61L 26/0004; A61L 26/0023; A61L 26/0066; A61L 26/008; A61L 2300/404

(57) ABSTRACT

The present invention relates to an antimicrobial hydrogel wound dressing composition comprising an electro-activated superoxidised solution comprising hypochlorous acid, sodium hypochlorite, and sodium chloride, and also sodium hyaluronate. The present invention also provides a use for said wound dressing composition for acute and chronic wounds, as well as method of preparation for making the same.

### FIELD OF INVENTION

The present invention relates to hydrogel compositions for management of acute and chronic wounds in human and animals.

### BACKGROUND OF THE INVENTION

Traditional wound management used in the medical field and first aid applications employ the use of antiseptic solutions and use of gauze and similar tools. As wound treatments demand quick treatment applications in order to treat wound without compromising infection, medical practitioners have shifted into the utility of gel based wound compositions, as gel compositions appear to be suitable for carrying antiseptic compositions.

International Patent Application WO201614892 presents a sheet-like crosslinked hyaluronate gel comprising hyaluronate based hydrogel and reticular fibers which is used for treating wounds with different levels of damage and exudates.

U.S. Pat. Application US20160114003 provides a pharmaceutical composition comprising collagen and sodium hyaluronate in the form of a hydrogel, pad, or dry spray, which is also applied in burns, bedsores, or chronic degenerative osteoarthritis, particularly also in applying a collagen-based dressing to the damaged epidermal areas or connective tissues helping to restore original native conditions.

U.S. Pat. Application US20200129564 provides an invention particularly an ointment, spray, gel, or hydrogel composition comprising an effective amount of chitosan, acetic acid, and chlorhexidine digluconate in an excipient for topical administration.

Additionally, many existing hydrogel compositions contain chlorhexidine, iodine, or silver which can create either antimicrobial resistance or toxicity within the subject being treated.

It appears that among similar wound dressing compositions in the market contains hypochlorous acid, and sodium hydrochloride which are commonly used in providing antimicrobial compositions, or only contains hyaluronate without having any antimicrobial agents. Therefore, in order to address and overcome existing problems in currently available wound dressings, there is a need to provide an antimicrobial wound dressing composition that has an antimicrobial property and also provides an enhanced would healing property.

### SUMMARY OF THE INVENTION

In one aspect, the invention provides an antimicrobial hydrogel wound dressing composition comprising an electro-activated superoxidised solution comprising hypochlorous acid, sodium hypochlorite, sodium chloride, and sodium hyaluronate.

In an aspect of the invention, antimicrobial hydrogel wound dressing composition comprises 0.001%-0.05% hypochlorous acid, 0.01-0.1 % sodium hypochlorite, 0.01 % -0.1% sodium chloride, 0.1%-5% sodium hyaluronate and 90%-99%- water. Said wound dressing composition further comprises thickening agent.

In an aspect of the invention, a use of an antimicrobial hydrogel wound dressing composition for managing acute and chronic wounds is provided. Said acute and chronic wounds are selected from the group consisting of diabetic foot ulcer, venous foot ulcer, pressure sore, burns, lacerations, cuts, bites, and abrasions. Said use further comprises use for managing surgical wounds. Surgical wounds are wounds derived from surgical procedures performed in a subject. Said use further comprises enhancing wound healing rate of a subject as compared to an average wound healing rate of a subject or providing enhanced formation of extracellular matrix within a wound area of a subject. Said use is further applied to human or animal subjects. Said further use of the present invention is for controlling pathogens or microbes within a wound area, particularly bacteria, fungi (such as yeast), bacterial spores, and fungal spores.

In an aspect of the invention, a use of an antimicrobial hydrogel wound dressing composition for managing moisture balance in wound is provided, or for administering or applying as a wound dressing prior to an application of an external wound dressing.

### DETAILED DESCRIPTION OF THE INVENTION

Further understanding of the object, construction, characteristics and functions of the invention, a detailed description with reference to the embodiments is given in the following.

The present invention provides an antimicrobial wound dressing composition comprising an electro-activated superoxidised solution comprising hypochlorous acid, sodium hypochlorite, and sodium chloride, and sodium hyaluronate.

In one embodiment, electro-activated superoxidised solution comprised in the wound dressing composition of the present invention is in the form of a pH stabilized electro-activated superoxidised solution. Said superoxidised solution comprises mainly of hypochlorous acid, sodium hypochlorite, and sodium chloride which are particularly stabilized in a range of pH 5.5 to pH 8.5.

In one embodiment, the superoxidised solution of the present invention is electro-activated by electrolysis of salt and water. Said superoxidised solution provides the antimicrobial property of the wound dressing composition, hence the antimicrobial agent solution. Said superoxidised solution prevents or minimizes wound infection in the localized area of the wound, specifically prevents growth or infection of pathogens such as bacterial pathogens. More specifically, hypochlorous acid and sodium hypochlorite are the antimicrobial active ingredients that target pathogens and microbes, particularly wound infective bacteria, fungi (such as yeast), bacterial spores, and fungal spores. The hypochlorous acid is neutral, thus can penetrate slime wall of microbes such as bacteria and creates osmotic shock that bursts the microbial cell leading to destruction of microbes. Unlike existing hydrogel compositions containing chlorhexidine, iodine, and silver that elicit antimicrobial resistance in the target microbes, and also toxicity in the subject being treated, the wound dressing of the present invention does not pose any risk for eliciting such adverse effects found in previous existing wound dressing technologies. Furthermore, the presence of both hypochlorous acid and sodium hypochlorite also prevents proliferation of microbes within the hydrogel, thus act as preservative components for the wound dressing composition or hydrogel wound dressing composition.

In one embodiment, the sodium hyaluronate comprised in the in the wound dressing composition of the present invention binds to the fibronectin in the wound which then aids in the enhanced or hastened formation or generation of the extracellular matrix around the wound as compared to a standard first-aid treated wound which uses materials such as sterile gauze in wound dressing. Having a rapid formation of an extracellular matrix, with sodium hyaluronate as catalyst, allows the wound to heal faster or the wound close up faster. Furthermore, sodium hyaluronate also provides an environment of elevated moisture for wound healing in a wound’s localized area as sodium hyaluronate is a hydrophilic agent which attracts moisture. An elevated moisture condition around the wound bed allows the fibronectin coming from the wound to move on an accessible environment around the would which contributes also to the faster formation of the extracellular matrix.

In another embodiment of the present invention is an antimicrobial wound dressing composition comprising,
an electro-activated superoxidised solution comprising,
0.001 to 0.05 % (w/v) hypochlorous acid,
0.01 to 0.1 % (w/v) sodium hypochlorite, and
0.01 to 0.1 % (w/v) sodium chloride;
0.1 to 5% (w/v) sodium hyaluronate; and
95 to 99 % (v/v) water.

In one embodiment, the antimicrobial wound dressing composition of the present invention is provided as a hydrogel or liquid sprayable solution. The invention’s provision as a hydrogel composition as a hydrogel retains more water or moisture content enhancing wound healing. The hydrogel form of the present invention is provided by utilizing gelling agents selected from the group consisting of Poly(acrylic acid) (Carbomer). However, it is most preferred that the gelling agent is Poly(acrylic acid) (Carbomer) as it is safe for topical use.

In one embodiment, it is preferred that the antimicrobial wound dressing composition of the present invention further comprises triethanolamine (TEA) for pH neutralization or pH adjustment. The amount of triethanolamine is preferred to be in 1.5 % (w/v).

In a preferred embodiment, an antimicrobial wound dressing composition of the present invention is packaged in tubes, spray bottles, aerosol containers, or any suitable containers for carrying the present invention.

In one embodiment, a use of an antimicrobial wound dressing composition for managing acute and chronic wounds is provided. Said acute and chronic wounds are selected from the group consisting of diabetic foot ulcer, venous foot ulcer, pressure sore, burns, lacerations, cuts, bites, and abrasions. Said use further comprises enhancing wound healing rate of a subject as compared to an average wound healing rate of a subject. Said use further comprises providing enhanced formation of extracellular matrix within a wound area of a subject. Said use for managing acute or chronic wound is intended to be administered to a subject, particularly a human or an animal having said wound. The present invention further provides use of an antimicrobial wound dressing composition for controlling pathogens and microbes within a wound area, preferably bacteria, fungi (such as yeast), bacterial spores, and fungal spores. The present invention further provides use of an antimicrobial wound dressing composition for managing moisture balance or moisture content in a wound or around a wound. Said use of the present invention is also for application or administration as applied wound dressing prior to an application of an external wound dressing.

### WORKING EXAMPLES

### Preparation of Antimicrobial Wound Dressing Composition

In an example provided, the antimicrobial hydrogel wound dressing composition is fabricated first by dissolving Poly(acrylic acid) (Carbomer) gelling agent in neutral hypochlorous acid solution. The mixing is done using stirrer or high shear mixer to homogenize the gel. The sodium hypochlorite is then added and mixing is done to ensure homogenous mix. The sodium hyaluronate is finally added into the mixture and mixing is done to achieve a homogenous gel with viscosity of between 7,000 to 12,000 mPas and final pH of between 5.5 to 8.5.

As would be apparent to a person having ordinary skilled in the art, the aforedescribed composition may be provided in many variations, modifications or alternatives relating to an antimicrobial hydrogel wound dressing composition, method and uses thereof. The principles and concepts disclosed herein may also be implemented in various manner which may not have been specifically described herein but which are to be understood as encompassed within the scope and letter of the following claims.

1. An antimicrobial wound dressing composition comprising:
an electro-activated superoxidised solution including:
hypochlorous acid,
sodium hypochlorite, and
sodium chloride; and
sodium hyaluronate.

2. The antimicrobial wound dressing composition of claim 1, comprising:
0.001 to 0.05 % hypochlorous acid;
0.01 to 0.1 % sodium hypochlorite;
0.01 to 0.1 % sodium chloride;
0.1 to 5% sodium hyaluronate; and
95% - 99% water.

3. The antimicrobial wound dressing composition of claim 1, further comprising triethanolamine (TEA) for neutralization.

4. The antimicrobial wound dressing composition of claim 1, further comprising a gelling agent.

5. The antimicrobial wound dressing composition of claim 1, wherein said composition as a pH of pH 5.5 to pH 8.5.

6. The antimicrobial wound dressing composition of claim 1, wherein said wound dressing composition is a gel composition, a hydrogel composition, or a sprayable liquid composition.

7. The antimicrobial wound dressing composition of claim 1, wherein said hypochlorous acid is sodium hypochlorite.

8. A use of the antimicrobial hydrogel wound dressing composition of claim 1 for managing acute wounds, chronic wounds, or surgical wounds.

9. The use of the antimicrobial hydrogel wound dressing composition for managing acute wounds, chronic wounds, or surgical wounds of claim 8, wherein:
said acute and chronic wounds are selected from the group consisting of diabetic foot ulcer, venous foot ulcer, pressure sore, burns, lacerations, cuts, bites, and abrasions; and
said surgical wounds are wounds derived after performing surgical procedure in the body of a subject.

10. The use of an antimicrobial hydrogel wound dressing composition for managing acute and chronic wounds of claim 9, comprising further use in enhancing wound healing rate of a subject as compared to an average wound healing rate of a subject.

11. The use of an antimicrobial hydrogel wound dressing composition for managing acute and chronic wounds of claim 10, wherein said subject is an animal or a human.

12. The use of an antimicrobial hydrogel wound dressing composition for managing acute and chronic wounds of claim 9, comprising further use providing enhanced formation of extracellular matrix within a wound area of a subject.

13. The use of an antimicrobial hydrogel wound dressing composition for managing acute and chronic wounds of claim 12, wherein said subject is an animal or a human.

14. A use of an antimicrobial hydrogel wound dressing composition of claim 1 for controlling pathogens and microbes within a wound area, pathogens and microbes said selected from the group consisting of bacteria, fungi, yeast, bacterial spores, and fungal spores.

15. A use of an antimicrobial hydrogel wound dressing composition of claim 1 for managing moisture balance in wound.

16. A use of an antimicrobial hydrogel wound dressing composition of claim 1 as an applied wound dressing prior to an application of an external wound dressing.
